(19) United States
(12) Patent Application Publication
(10) Pub. No.: US 2018/0169628 A1
(43) Pub. Date: Jun. 21, 2018

(54) CATALYST FOR THE DEHYDROGENATION OF HYDROCARBONS

(71) Applicant: BASF SE, Ludwigshafen (DE)

(72) Inventors: Florina Corina Patcas, Ludwigshafen (DE); Christophe Houssin, Lambsheim (DE); Martin Dieterle, Ludwigshafen (DE)

(21) Appl. No.: 15/834,460

(22) Filed: Dec. 7, 2017

Related U.S. Application Data
(62) Division of application No. 13/723,544, filed on Dec. 21, 2012.
(60) Provisional application No. 61/578,897, filed on Dec. 22, 2011.

(51) Int. Cl. B01J 23/889; C07C 5/32; C07C 5/333; C07C 15/46; C07C 11/167

(52) U.S. Cl. CPC B01J 23/8898; B01J 2523/842; C07C 5/322; B01J 2523/00; C07C 5/3332; C07C 2521/06; C07C 2523/04; C07C 2523/10; C07C 2523/34; C07C 2523/745; C07C 2523/84; C07C 15/46; C07C 11/167; B01J 2523/13; B01J 2523/22; B01J 2523/23; B01J 2523/3712; B01J 2523/47; B01J 2523/68; B01J 2523/72; B01J 23/8892

(57) ABSTRACT

The present invention relates to a catalyst for the dehydrogenation of hydrocarbons which is based on iron oxide and additionally comprises at least one potassium compound, at least one cerium compound, from 0.7 to 10% by weight of at least one manganese compound, calculated as Mn02, and from 10 to 200 ppm of at least one titanium compound, calculated as TiO2, and also to a process for the production thereof. Furthermore, the present invention relates to a process for the catalytic dehydrogenation of hydrocarbons using the catalyst of the invention.

### CROSS-REFERENCE TO RELATED APPLICATION

This application is a divisional application of U.S. Ser. No. 13/723,544, filed Dec. 21, 2012 which is a non-provisional application of U.S. Ser. No. 61/578,897, filed Dec. 22, 2011, all of which are incorporated herein by reference in their entirety.

The present invention relates to a catalyst for the dehydrogenation of hydrocarbons which is based on iron oxide and additionally comprises at least one potassium compound, at least one cerium compound, from 0.7 to 10% by weight of at least one manganese compound, calculated as MnO₂, and from 10 to 200 ppm of at least one titanium compound, calculated as TiO₂, and also to a process for the production thereof. Furthermore, the present invention relates to a process for the catalytic dehydrogenation of hydrocarbons using the catalyst of the invention.

The use of iron oxide-based dehydrogenation catalysts in the dehydrogenation of various hydrocarbons to the corresponding unsaturated hydrocarbons has long been known in the prior art. For example, the dehydrogenation of ethylbenzene to styrene, isopropylbenzene to alpha-methylstyrene, butene to butadiene or isoamylene to isoprene are of industrial importance. The preparation of styrene by heterogeneously catalyzed dehydrogenation of ethylbenzene in the presence of steam is a process which has been carried out industrially since the beginning of the 1930s and has become established as a synthetic route to styrene. Styrene is one of the most important monomers of the plastics industry and is used, for example, for preparing polystyrene, acrylonitrile-butadiene-styrene polymer (ABS) and synthetic rubber.

The iron oxide-based dehydrogenation catalysts described in the prior art are generally multicomponent systems and comprise essentially iron oxide and an alkali metal compound which is, for example, used as alkali metal oxide, carbonate or hydroxide in the production of the catalyst. In addition, these catalysts generally comprise various further active components (promoters), for example oxides of the elements of transition groups 5 and 6 of the Periodic Table or of the rare earths.

The catalytic dehydrogenation of aliphatic or alkylaromatic hydrocarbons is usually carried out industrially in the presence of steam at temperatures in the range from 500 to 700° C. In these processes, the hydrocarbon and the steam are mixed and passed over the iron oxide dehydrogenation catalyst at elevated temperatures.

During the course of the dehydrogenation process, the active sites of the dehydrogenation catalyst typically become blocked as a result of the formation of carbonaceous material during the catalytic dehydrogenation (for example of ethylbenzene to styrene) and gradual deactivation of the catalyst occurs. To reduce this deactivation, steam is added to the hydrocarbon. The steam enables the carbonaceous material formed on the catalyst surface to be gasified in-situ, thus enabling the active catalyst surface to be regenerated. In addition, the steam typically has the following additional functions: supplying the heat of reaction required for the endothermic dehydrogenation reaction, shifting the equilibrium to the product side by reducing the partial pressures of the starting materials, maintaining the oxidation state of the iron notwithstanding the reducing action of hydrogen and hydrocarbon.

The stability and activity of the catalyst are generally higher, the higher the ratio of steam to hydrocarbon (S/HC ratio). However, from the point of view of energy consumption and the operating costs associated therewith, it is desirable to reduce the steam/hydrocarbon ratio. However, a low steam/hydrocarbon ratio typically increases carbonization and irreversible reduction of the dehydrogenation catalyst, so that the catalyst activity decreases after a relatively short time. In addition, a certain amount of steam is normally necessary to supply the required energy to the reaction system.

To ensure satisfactory operating periods of the dehydrogenation process at a low steam/hydrocarbon ratio, the catalysts have to meet particular requirements. In the catalytic dehydrogenation of hydrocarbons, a molar steam/hydrocarbon ratio of less than or equal to 7.35 is generally referred to as a low S/HC ratio. In the case of the dehydrogenation of ethylbenzene, this corresponds approximately to a weight ratio of steam/hydrocarbon of less than or equal to 1.25.

Numerous dehydrogenation catalysts based on iron oxide have been described in the prior art. EP-A 0 181 999 describes dehydrogenation catalysts comprising iron oxide, potassium oxide, magnesium oxide and optionally further metal compounds. The optional addition of from 0 to 10% by weight of a compound Me₂O₃ where Me=Cr or Mn is described, inter alia. Document EP-A 0 181 999 does not disclose any examples of a dehydrogenation catalyst comprising a manganese compound. In addition, the addition of titanium is not described. The catalyst described in EP-A 0 181 999 is said to have, in particular, an improved stability to boiling water.

The document WO 96/18457 describes a restructured iron oxide having specific particle properties and its use in dehydrogenation catalysts. The restructured iron oxide is said to be obtained by reacting an iron oxide with a restructuring agent, with the restructuring agent being able to be selected, in particular, from among compounds of molybdenum, copper, calcium, zinc, cobalt and cerium. The document WO 96/18457 describes the use of these catalysts in dehydrogenation processes at moderate to high molar S/HC ratios of about 10.

The document EP-B 0 956 899 describes a dehydrogenation catalyst comprising iron oxide, potassium oxide, magnesium oxide, a further metal oxide and at least two rare earth metal oxides. Many further promoters can optionally be comprised, including, for example, MnO₃. The addition of from 10 to 200 ppm of at least one titanium compound is not described in EP-B 0 956 899. The document EP-B 0 956 899 also describes the use of catalysts for the dehydrogenation of alkylaromatic compounds at a weight ratio of steam/hydrocarbon in the range from 0.5 to 2.5.

The document EP-A 0 502 510 relates to dehydrogenation catalysts comprising iron oxide, potassium oxide and from 0.005 to 0.95% by weight of titanium oxide as significant constituents. In addition, further promoters, in particular cerium oxide, molybdenum oxide or magnesium oxide, can be comprised. The addition of manganese is not described.

The document U.S. Pat. No. 4,220,560 describes a dehydrogenation catalyst comprising an iron-chromium spinel, which additionally comprises a further metal selected from among cobalt, zinc, manganese and magnesium. In addition, the use of the catalyst for the dehydrogenation of hydrocarbons at a molar steam/hydrocarbon ratio of from 9 to 12 is described. The addition of titanium is not described in the document U.S. Pat. No. 4,220,560.

The document US 2006/0106267 discloses a catalyst and its use for preparing styrene at a steam/hydrocarbon weight ratio of less than or equal to 1.35. The catalyst described in the document US 2006/0106267 comprises iron oxide together with a cerium compound, a potassium compound, a molybdenum compound, an alkaline earth metal compound and a small proportion of titanium dioxide. Apart from titanium dioxide, further promoters can be comprised. The addition of from 0.7 to 10% by weight of a manganese compound is not described. The document US 2006/0106:267 states that a styrene catalyst having a very low titanium content should have the greatest stability.

The document WO 99/49966 describes a catalyst which comprises iron oxide, potassium oxide, a magnesium compound and a cerium compound and has an iron-potassium phase, and also its use in the dehydrogenation of ethylbenzene. No catalyst comprising a titanium compound and from 0.7 to 10% by weight of a manganese compound is described.

The addition of manganese to dehydrogenation catalysts based on iron oxide is described, for example, in the publications Miyakoshi et al. (Appl. Cat. A 216, 2001, pp. 137-146) and Kotarba et al. (J. Cat. 221, 2004, pp. 650-652). Miyakoshi et al. describe a sol-gel process for producing an iron oxide dehydrogenation catalyst, with an MnFe2O₄ spinel being said to be formed in the catalyst. Kotarba et al. describe a manganese-doped iron oxide catalyst comprising an active K₂Fe₂₂O₃₄ ferrite phase, with the loss of potassium from the catalyst being said to be reduced by means of the manganese doping.

The document Liao et al. (Cat. Comm. 9, 2008, pp. 1817-1821) describes the influence of the addition of titanium dioxide on the structure and reactivity of an iron oxide dehydrogenation catalyst. An iron-potassium dehydrogenation catalyst to which from 1000 to 15 000 ppm of titanium dioxide have been added is described.

None of the documents discloses a positive effect of manganese in combination with titanium in a dehydrogenation catalyst comprising an iron compound, a potassium compound and a cerium compound. In addition, none of the documents discloses a positive effect of manganese and/or titanium on the catalytic performance at a low molar steam/hydrocarbon ratio of less than 7.35 (mol/mol).

There is a need for further-improved dehydrogenation catalysts for the dehydrogenation of hydrocarbons, which catalysts have increased stability at improved or equal catalyst activity and thus higher operating lives. It is an object of the present invention to provide an improved dehydrogenation catalyst based on iron oxide which displays, in particular, improved stability and/or an improved catalyst activity.

A further object of the present invention is to provide improved dehydrogenation catalysts which display better stability and/or activity than the catalyst compositions of the prior art in dehydrogenation processes at a low steam/hydrocarbon ratio (S/HC ratio), i.e. in particular at molar S/HC ratios of less than or equal to 7.35.

Likewise, a satisfactory mechanical stability of the catalyst and resistance to boiling water should be ensured. In addition, the production of the dehydrogenation catalyst should be able to be carried out simply and inexpensively, and in particular no complicated process steps, for example a sol-gel process, and/or high calcination temperatures should be necessary in the production of the catalyst.

It has now surprisingly been found that dehydrogenation catalysts comprising from 0.7 to 10% by weight of a manganese compound can be used advantageously, i.e. with a satisfactory operating life and high yields, in dehydrogenation processes at a low S/HC ratio (molar S/HC ratio of less than or equal to 7.35, in particular less than or equal to 6).

It has also been found that the promoting effect of manganese can, in particular, be increased by addition of small amounts of titanium in dehydrogenation catalysts comprising iron oxide, a potassium compound, a cerium compound and a manganese compound. In this context, it has been possible to determine an optimal amount of titanium. It has been found that the promoting effect of manganese can be changed into an inhibiting effect when the amount of titanium is increased.

The invention provides a dehydrogenation catalyst comprising at least one iron compound, at least one potassium compound, at least one cerium compound, from 0.7 to 10% by weight, preferably from 0.7 to 5% by weight, particularly preferably from 0.7 to 3% by weight, in particular from 0.7 to 2% by weight, in particular from 1 to 2% by weight, of at least one manganese compound, calculated as MnO₂, and from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂.

Unless indicated otherwise, all the following figures in % by weight are based on the total dehydrogenation catalyst and are in each case calculated for the metal oxide in the highest oxidation state. For the purposes of the present invention, ppm means milligram per kilogram (mg/kg).

The catalysts of the invention display an improved activity and stability compared to the catalysts described in the prior art. In particular, the catalysts of the invention display improved properties in dehydrogenation processes at a low steam/hydrocarbon ratio (S/HC ratio), i.e. at molar S/HC ratios of less than or equal to 7.35. This improved activity at a low S/HC ratio is also reflected, for example, in a reduced decrease in the activity compared to known catalysts when a change is made from moderate to low S/HC ratios.

The expression “dehydrogenation catalyst comprising at least one iron compound, at least one potassium compound, at least one cerium compound and at least one manganese compound and optionally further metal compounds” means, for the purposes of the present invention, that the corresponding metals can be determined in the optionally indicated amounts in the catalyst. Mixed phases (e.g. oxide mixed phases) and/or isolated phases of the metal compounds described below can typically be present in the catalyst. It is also possible for one or more of the component(s) described below to be comprised, partly or completely, in another raw material used in production of the catalyst.

According to the invention, the dehydrogenation catalyst comprises at least one iron compound or at least one iron compound is used in the production of the dehydrogenation catalyst. The at least one iron compound is preferably an iron oxide, in particular Fe₂O₃.

The at least one iron compound is preferably selected from among natural or synthetic iron oxides and/or iron oxide hydroxides. In particular, the at least one iron compound is selected from the group consisting of a-Fe₂O₃ (hematite), y-Fe₂O₃, iron oxide hydroxide (e.g. α-FeOOH, goethite) and Fe₃O₄ (magnetite). As synthetic iron oxides, it is possible to use, for example, iron oxides which have been prepared by thermal decomposition of iron salt solutions.

In particular, the at least one iron compound and the at least one potassium compound can be present in the form of a potassium ferrite phase KₓFe_{y}O_{z}, (where x=1; 2, y=1-22 and z=2-34).

Preference is given to using α-Fe₂O₃ (hematite) as iron compound. The use of α-Fe₂O₃ (hematite) in combination with goethite (FeOOH) and/or magnetite (Fe₃O₄) as iron compound is also preferred. The proportion of goethite (FeOOH) and/or magnetite (Fe₃O₄) is then typically from 0 to 30% by weight (based on the total amount of iron compound).

The specific surface area of the iron compound (e.g. as determined by the BET method) is typically in the range from 1 to 50 m²/g, preferably from 1 to 20 m²/g.

The at least one iron compound is typically comprised in an amount in the range from 50 to 90% by weight, preferably from 60 to 80% by weight, particularly preferably from 65 to 75% by weight, calculated as Fe₂O₃, in the dehydrogenation catalyst (based on the total weight of the dehydrogenation catalyst).

According to the invention, the catalyst comprises at least one potassium compound or at least one potassium compound is used in the production of the dehydrogenation catalyst. The at least one potassium compound is preferably selected from among potassium oxide, potassium carbonate, potassium hydroxide, potassium hydrogencarbonate, potassium oxalate and potassium ferrite, in particular selected from among potassium oxide, potassium carbonate, potassium hydroxide and potassium hydrogencarbonate. The at least one potassium compound is in particular a potassium oxide (K₂O) or a mixed oxide. It is also possible to use another thermally decomposable potassium compound. The at least one potassium compound can typically be present in the catalyst as oxide mixed phase with the metals present in the catalyst.

The at least one potassium compound is typically comprised in an amount in the range from 1 to 30% by weight, preferably from 5 to 25% by weight, in particular from 10 to 15% by weight, in the dehydrogenation catalyst (based on the total weight of the dehydrogenation catalyst and calculated as K₂O).

According to the invention, the dehydrogenation catalyst comprises at least one cerium compound or at least one cerium compound is used in the production of the dehydrogenation catalyst.

The at least one cerium compound is preferably selected from among cerium oxides, cerium hydroxides, cerium carbonates, water-comprising cerium carbonate and cerium oxalates. Preference is given to using mixtures of the cerium compounds mentioned. The at least one cerium compound is preferably selected from among cerium(IV) oxide (CeO₂), cerium(III) oxalate and cerium(III) carbonate, preferably from among cerium(IV) oxide (CeO₂) and cerium(III) carbonate. The at least one cerium compound is typically converted into cerium dioxide in the production of the catalyst.

The dehydrogenation catalyst preferably comprises from 2 to 20% by weight, preferably from 5 to 15% by weight, in particular from 5 to 10% by weight, of at least one cerium compound, calculated as CeO₂. According to the invention, the catalyst comprises from 0.7 to 10% by weight of at least one manganese compound, calculated as MnO₂; or at least one manganese compound is used in the amount indicated in the production of the dehydrogenation catalyst. The at least one manganese compound is preferably selected from among manganese oxides (e.g. MnO, Mn₂O₃, MnO₂, Mn₃O₄), manganese carbonates and permanganates. The at least one manganese compound is particularly preferably selected from the group consisting of MnO, Mn₂O₃, MnO₂ (e.g. pyrolusite), Mn₃O₄ and Mn₂O₇. In particular, the at least one manganese compound is a manganese oxide, in particular MnO₂.

The at least one manganese compound is preferably added as manganese dioxide (MnO₂) in the production of the catalyst. However, it is also possible to use other oxides of manganese or else other manganese compounds which decompose thermally. Furthermore, it is possible for the at least one manganese compound to be comprised partially or completely in another raw material used in production of the catalyst, e.g. in the iron oxide.

The manganese comprised in the catalyst is preferably not present in the form of a manganese-iron mixed oxide. At least 80% by weight, preferably at least 90% by weight, (based on the total amount of manganese) of the manganese comprised in the catalyst is preferably not present in the form of a manganese-iron mixed oxide. The manganese in the present catalyst preferably partly or virtually completely forms an independent manganese oxide phase. It is also possible for the manganese comprised in the catalyst to be partly or virtually completely present in the form of a manganese-potassium mixed oxide phase.

According to the invention, the dehydrogenation catalyst comprises from 0.7 to 10% by weight, preferably from 0.7 to 5% by weight, particularly preferably from 0.7 to 3% by weight, in particular from 0.7 to 2% by weight, in particular from 1 to 2% by weight, of at least one manganese compound, calculated as MnO₂ (based on the total catalyst).

According to the invention, the dehydrogenation catalyst comprises from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂, or at least one titanium compound is used in the production of the dehydrogenation catalyst.

For the purposes of the present invention, ppm is milligram per kilogram (mg/kg).

The at least one titanium compound can, in particular, be selected from among titanium oxides, titanium alkoxides and titanium carboxylates. The at least one titanium compound is preferably titanium dioxide (TiO₂). The at least one titanium compound is preferably added as titanium dioxide (TiO₂) in the production of the catalyst. However, it is also possible to use other titanium compounds. Furthermore, it is possible for the at least one titanium compound to be comprised partly or completely in another raw material used in production of the catalyst, e.g. in the iron oxide.

It has been found that it is possible to produce a catalyst having particularly advantageous properties by means of, in particular, the combination of the at least one manganese compound and the at least one titanium compound, with, in particular, a synergistic effect being observed in respect of improved activity and yield. The addition of from 10 to 200 ppm of at least one titanium compound, calculated as TiO₂, has been found to be particularly advantageous. In addition, it has been found that the addition of titanium without the addition of manganese brings about a significantly smaller improvement in the catalyst activity.

A preferred embodiment of the present invention therefore provides a dehydrogenation catalyst comprising from 0.7 to 10% by weight, preferably from 0.7 to 5% by weight, particularly preferably from 0.7 to 3% by weight, in particular from 0.7 to 2% by weight, in particular from 1 to 2% by weight, of at least one manganese compound, calculated as MnO₂, and from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂.

In a particularly preferred embodiment, the present invention provides a dehydrogenation catalyst comprising from 0.7 to 3% by weight of at least one manganese compound, calculated as MnO₂, and from 50 to 120 ppm of at least one titanium compound, calculated as TiO₂.

In a particularly preferred embodiment, the present invention provides a dehydrogenation catalyst comprising from 0.7 to 2% by weight of at least one manganese compound, calculated as MnO₂, and from 60 to 100 ppm of at least one titanium compound, calculated as TiO₂.

In a preferred embodiment, the present invention relates to a dehydrogenation catalyst as described above, comprising:
from 50 to 90% by weight of at least one iron compound, calculated as Fe₂O₃; from 1 to 30% by weight of at least one potassium compound, calculated as K₂O;
from 0.7 to 10% by weight, preferably from 0.7 to 5% by weight, particularly preferably from 0.7 to 3% by weight, in particular from 0.7 to 2% by weight, in particular from 1 to 2% by weight, of at least one manganese compound, calculated as MnO₂;
from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂;
from 2 to 20% by weight of at least one cerium compound, calculated as CeO₂; and optionally from 0 to 30% by weight of at least one further component.

In a preferred embodiment, the abovementioned components add up to 100% by weight.

The further components can be comprised (or be added) in amounts of from 0 to 30% by weight, preferably from 0 to 20% by weight, more preferably from 0.001 to 10% by weight, in particular from 0.1 to 5% by weight, in particular from 0.5 to 5% by weight.

The dehydrogenation catalyst can preferably comprise at least one compound selected from the group consisting of molybdenum (Mo), tungsten (W) and vanadium (V) as further component; or at least one such compound is added in the production of the dehydrogenation catalyst. The further component can, in particular, be selected from among oxygen compounds (for example oxides, oxide hydrates, oxo compounds) of molybdenum (Mo), tungsten (W) and vanadium (V). In particular, the at least one compound selected from the group consisting of molybdenum (Mo), tungsten (W) and vanadium (V) is a compound which is decomposed thermally in the production of the dehydrogenation catalyst.

Preference is given to using at least one molybdenum compound selected from among molybdenum oxides and molybdates (e.g. ammonium molybdate, potassium molybdate) as at least one further component. The at least one molybdenum compound is preferably ammonium heptamolybdate.

The dehydrogenation catalyst preferably comprises from 0.1 to 10% by weight, preferably from 1 to 5% by weight, of at least one compound selected from the group consisting of molybdenum, tungsten and vanadium, calculated as oxide in the respective highest oxidation state, as further component.

In particular, the dehydrogenation catalyst comprises from 0.1 to 10% by weight, preferably from 1 to 5% by weight, of at least one molybdenum compound, calculated as MoO₃, as further component.

Furthermore, the dehydrogenation catalyst can comprise from 0 to 10% by weight, particularly preferably from 1 to 5% by weight, of at least one vanadium compound, calculated as V₂O₅.

The dehydrogenation catalyst preferably comprises at least one alkaline earth metal compound as further component, or at least one alkaline earth metal compound is used in the production of the dehydrogenation catalyst. In particular, the dehydrogenation catalyst can comprise from 0.1 to 10% by weight, preferably from 1 to 5% by weight, of at least one alkaline earth metal compound, calculated as oxide, as further component.

In a preferred embodiment, the dehydrogenation catalyst comprises at least one magnesium compound as further component. The dehydrogenation catalyst preferably comprises from 0.1 to 10% by weight, preferably from 1 to 5% by weight, of at least one magnesium compound, calculated as MgO, as further component. In particular, the at least one magnesium compound is selected from among magnesium oxide, magnesium carbonate (e.g. magnesite) and magnesium hydroxide. The at least one magnesium compound is preferably magnesium oxide (MgO) and/or magnesium carbonate (MgCO₃) (e.g. magnesite). Preference is given to using magnesium oxide (MgO) and/or magnesium carbonate (MgCO₃) (e.g. magnesite) as further component in the production of the catalyst.

In a preferred embodiment, the dehydrogenation catalyst comprises at least one calcium compound as further component. The dehydrogenation catalyst preferably comprises from 0.1 to 10% by weight, preferably from 1 to 5% by weight, of at least one calcium compound, calculated as CaO, as further component. In particular, the at least one calcium compound is selected from among calcium oxide, calcium carbonate and calcium hydroxide. The at least one calcium compound is preferably calcium oxide (CaO). Preference is given to using calcium oxide (CaO) and/or calcium hydroxide (Ca(OH)₂) as further component in the production of the catalyst.

In a preferred embodiment, the dehydrogenation catalyst comprises at least one magnesium compound and at least one calcium compound. In particular, the dehydrogenation catalyst comprises from 0.1 to 10% by weight, preferably from 1 to 5% by weight, of at least one magnesium compound, calculated as MgO, and from 0.1 to 10% by weight, preferably from 1 to 5% by weight, of at least one calcium compound, calculated as CaO.

In a preferred embodiment, the above-described dehydrogenation catalyst comprises, as further components:
from 0.1 to 10% by weight, preferably from 1 to 5% by weight, of at least one alkaline earth metal compound, calculated as oxide, and
from 0.1 to 10% by weight, preferably from 1 to 5% by weight, of at least one compound selected from the group consisting of molybdenum (Mo), tungsten (W) and vanadium (V) (in each case calculated as the oxide in the highest oxidation state).

In a further preferred embodiment, the above-described dehydrogenation catalyst comprises, as further components:
from 0.1 to 10% by weight, preferably from 1 to 5% by weight, of at least one magnesium compound, calculated as MgO;
from 0.1 to 10% by weight, preferably from 1 to 5% by weight, of at least one calcium compound, calculated as CaO;
from 0.1 to 10% by weight, preferably from 1 to 5% by weight, of at least one molybdenum compound, calculated as MoO₃.

Furthermore, the dehydrogenation catalyst can comprise one or more of the customary compounds for increasing the activity and/or selectivity, for example compounds selected from among Cr, Co, Ni, Cu, Zn, Al, Ga, Ge, Zr, Nb, Ru, Rh, Pd, Ag, Cd, In, Sn, Sb, La, Hf, Ta, Re, Ir, Pt, Au, Pb and Bi, as at least one further component (as promoter or dopant). The abovementioned customary promoters can typically be comprised in amounts of from 0 to 10% by weight, preferably from 0.001 to 5% by weight, preferably from 0.01 to 2% by weight.

In an embodiment, the above-described dehydrogenation catalyst comprises at least one further rare earth metal compound apart from cerium, in particular selected from the group consisting of lanthanum (La), praseodymium (Pr) and neodymium (Nd), as further component. The dehydrogenation catalyst preferably comprises from 1 to 1000 ppm, preferably from 10 to 500 ppm, particularly preferably from 20 to 300 ppm, of at least one further rare earth metal compound apart from cerium, calculated as oxide in the respective highest oxidation state. In particular, the catalyst comprises from 1 to 1000 ppm, preferably from 10 to 500 ppm, particularly preferably from 20 to 300 ppm, of at least one rare earth metal compound selected from the group consisting of lanthanum, praseodymium and neodymium. The dehydrogenation catalyst can preferably comprise from 1 to 1000 ppm, preferably from 3 to 500 ppm, particularly preferably from 10 to 100 ppm, of at least one lanthanum compound, calculated as La₂O₃, as further component. The dehydrogenation catalyst can preferably comprise from 1 to 1000 ppm, preferably from 3 to 500 ppm, particularly preferably from 10 to 100 ppm, of at least one praseodymium compound, calculated as PrO₂, as further component. The dehydrogenation catalyst can preferably comprise from 1 to 1000 ppm, preferably from 3 to 500 ppm, particularly preferably from 10 to 100 ppm, of at least one neodymium compound, calculated as Nd₂O₃, as further component.

The above-described dehydrogenation catalyst can preferably comprise at least one compound of metals of transition groups 8 to 12 of the Periodic Table of the Elements as further component. The above-described dehydrogenation catalyst preferably comprises at least one compound of metals selected from the group consisting of ruthenium (Ru), osmium (Os), cobalt (Co), nickel (Ni), palladium (Pd), platinum (Pt), copper (Cu), silver (Ag), gold (Au) and zinc (Zn); preferably selected from the group consisting of cobalt (Co), nickel (Ni), palladium (Pd), copper (Cu), and zinc (Zn); particularly preferably selected from the group consisting of nickel (Ni), copper (Cu), and zinc (Zn), as further component. The above-described dehydrogenation catalyst can, in particular, comprise from 1 to 1000 ppm, preferably from 50 to 500 ppm, particularly preferably from 50 to 200 ppm, of at least one compound of metals of transition groups 8 to 12 of the Periodic Table of the Elements, in each case calculated as oxide in the highest oxidation state, as further component. In a preferred embodiment, the above-described dehydrogenation catalyst comprises from 1 to 1000 ppm, preferably from 50 to 500 ppm, particularly preferably from 50 to 200 ppm, of at least one compound of metals selected from the group consisting of nickel (Ni), copper (Cu), and zinc (Zn), in each case calculated as oxide in the highest oxidation state. The dehydrogenation catalyst can preferably comprise from 1 to 1000 ppm, preferably from 30 to 500 ppm, particularly preferably from 30 to 200 ppm, of at least one nickel compound, calculated as NiO, as further component. The dehydrogenation catalyst can preferably comprise from 1 to 1000 ppm, preferably from 10 to 200 ppm, particularly preferably from 30 to 100 ppm, of at least one copper compound, calculated as CuO, as further component. The dehydrogenation catalyst can preferably comprise from 1 to 1000 ppm, preferably from 1 to 500 ppm, particularly preferably from 10 to 100 ppm, of at least one zinc compound, calculated as ZnO, as further component.

In addition, the dehydrogenation catalyst can comprise at least one compound of elements of main group 4 of the Periodic Table of the Elements as further component. The above-described dehydrogenation catalyst preferably comprises at least one compound selected from the group consisting of silicon (Si), germanium (Ge), tin (Sn) and lead (Pb) compounds, preferably at least one silicon compound, as further component. In particular, the dehydrogenation catalyst comprises from 1 to 1000 ppm, preferably from 5 to 500 ppm, particularly preferably from 10 to 100 ppm, of at least one compound selected from the group consisting of silicon (Si), germanium (Ge), tin (Sn) and lead (Pb) compounds, calculated as oxide in the respective highest oxidation state. In an embodiment, the dehydrogenation catalyst described comprises from 1 to 1000 ppm, preferably from 5 to 500 ppm, particularly preferably from 10 to 100 ppm, of at least one silicon compound, calculated as SiO₂.

The above-described dehydrogenation catalyst can typically comprise at least one nonmetal selected from among nonmetals of main groups 5 to 7 of the Periodic Table of the Elements, in particular selected from the group consisting of nitrogen, phosphorus, sulfur and chlorine, as nonmetal in addition to oxygen.

In a preferred embodiment, the dehydrogenation catalyst comprises:
from 50 to 90% by weight, particularly preferably from 60 to 80% by weight, of at least one iron compound, calculated as Fe₂O₃;
from 1 to 30% by weight, particularly preferably from 5 to 25% by weight, of at least one potassium compound, calculated as K₂O;
from 0.7 to 10% by weight, preferably from 0.7 to 5% by weight, particularly preferably from 0.7 to 3% by weight, in particular from 0.7 to 2% by weight, particularly preferably from 1 to 2% by weight, of at least one manganese compound, calculated as MnO₂;
from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂;
from 2 to 20% by weight, particularly preferably from 5 to 15% by weight, of at least one cerium compound, calculated as CeO₂;
from 0.1 to 10% by weight, particularly preferably from 1 to 5% by weight, of at least one magnesium compound, calculated as MgO;
from 0.1 to 10% by weight, particularly preferably from 1 to 5% by weight, of at least one calcium compound, calculated as CaO;
from 0.1 to 10% by weight, particularly preferably from 1 to 5% by weight, of at least one molybdenum compound, calculated as MoO₃;
from 0 to 10% by weight, particularly preferably from 1 to 5% by weight, of at least one vanadium compound, calculated as V₂O₅, and
from 0 to 10% by weight of at least one further component.

In a preferred embodiment, the abovementioned components add up to 100% by weight.

In a preferred embodiment, the dehydrogenation catalyst comprises:
from 50 to 90% by weight, particularly preferably from 60 to 80% by weight, of at least one iron compound, calculated as Fe₂O₃;
from 1 to 30% by weight, particularly preferably from 5 to 25% by weight, of at least one potassium compound, calculated as K₂O;
from 0.7 to 10% by weight, preferably from 0.7 to 5% by weight, particularly preferably from 0.7 to 3% by weight, in particular from 0.7 to 2% by weight, particularly preferably from 1 to 2% by weight, of at least one manganese compound, calculated as MnO₂;
from 10 to 200 ppm, preferably from 30 to 150 ppm, particularly preferably from 50 to 120 ppm, in particular from 60 to 100 ppm, very particularly preferably from 60 to 80 ppm, of at least one titanium compound, calculated as TiO₂;
from 2 to 20% by weight, particularly preferably from 5 to 15% by weight, of at least one cerium compound, calculated as CeO₂;
from 0.1 to 10% by weight, particularly preferably from 1 to 5% by weight, of at least one magnesium compound, calculated as MgO;
from 0.1 to 10% by weight, particularly preferably from 1 to 5% by weight, of at least one calcium compound, calculated as CaO;
from 0.1 to 10% by weight, particularly preferably from 1 to 5% by weight, of at least one molybdenum compound, calculated as MoO₃;
from 0 to 10% by weight, particularly preferably from 1 to 5% by weight, of at least one vanadium compound, calculated as V₂O₅, and
from 1 to 10 000 ppm, preferably from 10 to 5000 ppm, in particular from 10 to 3000 ppm, of at least one further component selected from among lanthanum compounds, praseodymium compounds, neodymium compounds, nickel compounds, copper compounds, zinc compounds and silicon compounds, in each case calculated as oxide in the highest oxidation state.

In a preferred embodiment, the abovementioned components add up to 100% by weight.

All figures in % by weight are based, unless indicated otherwise, on the total dehydrogenation catalyst. All figures in % by weight were, unless indicated otherwise, calculated as oxide of the metal concerned, in each case in the highest oxidation state.

In particular, the present invention provides an above-described dehydrogenation catalyst for the catalytic dehydrogenation of hydrocarbons at a molar steam/hydrocarbon weight ratio in the range from 3 to 7.35; preferably in the range from 4 to 7; in particular in the range from 5 to 6.

Furthermore, the present invention provides a process for producing a dehydrogenation catalyst as described above, which comprises the following steps:
i) production of a catalyst premix by mixing at least one iron compound, at least one potassium compound, at least one cerium compound, from 0.7 to 10% by weight, based on the finished catalyst, of at least one manganese compound, calculated as MnO₂, from 10 to 200 ppm, based on the finished catalyst, of at least one titanium compound, calculated as TiO₂, optionally further metal compounds, optionally further components and optionally at least one binder with a solvent;
ii) production of shaped catalyst bodies from the catalyst premix obtained in step i);
iii) drying of the shaped catalyst bodies and calcination of the shaped catalyst bodies.

The basic mode of operation in the production of dehydrogenation catalysts is known to those skilled in the art. The above-described dehydrogenation catalysts can be produced, for example, as described in WO 99/49966.

In the process for producing a dehydrogenation catalyst, preference is given to using the metal compounds and further components described above in connection with the dehydrogenation catalyst. In particular, the above-described iron compounds, potassium compounds, cerium compounds, manganese compounds and titanium compounds are used in the production of the dehydrogenation catalyst. The above-described, in particular, molybdenum compounds, vanadium compounds, magnesium compounds and calcium compounds can optionally be used in the production of the dehydrogenation catalyst. The above-described further metal compounds can optionally be used in the production of the catalyst, and preference is given to one or more of the further metal compounds being entirely or partly present in one of the raw materials used, in particular in the iron oxide and/or cerium carbonate.

As compounds and further components, it is possible to use compounds as they are present in the finished catalyst or compounds which are converted during the production process into compounds as are present in the finished catalyst.

In particular, the invention provides a process for producing a dehydrogenation catalyst as described, wherein an iron(III) oxide (Fe₂O₃) is used as at least one iron component. In particular, it is possible to use an iron oxide having the following composition, where the figures indicate the amount of the element or the compounds based on the total amount of iron oxide:
from 95 to 99.99% by weight, preferably from 98 to 99.99% by weight, of iron(III) oxide (Fe₂O₃);
from 1 to 10 000 ppm, preferably from 1 to 5000 ppm, particularly preferably from 1 to 1000 ppm, of manganese (Mn);
from 0 to 1000 ppm, preferably from 1 to 500 ppm, particularly preferably from 1 to 100 ppm, of sodium (Na);
from 0 to 1000 ppm, preferably from 1 to 500 ppm, particularly preferably from 1 to 50 ppm, of calcium (Ca);
from 0 to 1000 ppm, preferably from 1 to 500 ppm, particularly preferably from 1 to 100 ppm, of copper (Cu);
from 0 to 1000 ppm, preferably from 1 to 500 ppm, particularly preferably from 1 to 100 ppm, of nickel (Ni);
from 0 to 1000 ppm, preferably from 1 to 500 ppm, particularly preferably from 1 to 100 ppm, of zinc (Zn);
from 0 to 300 ppm, preferably from 1 to 200 ppm, particularly preferably from 1 to 100 ppm, of titanium (Ti);
from 0 to 1000 ppm, preferably from 1 to 500 ppm, particularly preferably from 1 to 100 ppm, of chromium (Cr);
from 0 to 1000 ppm, preferably from 1 to 500 ppm, particularly preferably from 1 to 100 ppm, of silicon (Si);
from 0 to 1000 ppm, preferably from 1 to 500 ppm, particularly preferably from 1 to 100 ppm of chlorine (Cl);
from 0 to 10 000 ppm, preferably from 1 to 5000 ppm, particularly preferably from 1 to 1000 ppm, of sulfur (S).

In particular, the invention provides a process for producing a dehydrogenation catalyst as described, wherein a potassium carbonate (K₂CO₃) is used as at least one potassium compound. In particular, it is possible to use a potassium carbonate having the following composition, where the figures indicate the amount of the element or the compounds based on the total amount of potassium carbonate:
from 80 to 99% by weight, preferably from 80 to 85% by weight, of potassium carbonate;
from 0 to 1% by weight, preferably from 0.01 to 1% by weight, preferably from 0.01 to 0.5% by weight, of sodium (Na);
from 0 to 100 ppm, preferably from 1 to 50 ppm, particularly preferably from 1 to 20 ppm, of iron (Fe);
from 0 to 100 ppm, preferably from 1 to 50 ppm, of chlorine (Cl).

In particular, the invention provides a process for producing a dehydrogenation catalyst as described, wherein a cerium(III) carbonate (CeCO₃) is used as at least one cerium compound. In particular, it is possible to use a cerium carbonate having the following composition, where the figures indicate the amount of the element or the compounds based on the total amount of cerium carbonate:
from 40 to 85% by weight, preferably from 45 to 65% by weight, of CeO₂;
from 1 to 1000 ppm, preferably from 100 to 500 ppm, particularly preferably from 100 to 300 ppm, of lanthanum (La);
from 1 to 1000 ppm, preferably from 100 to 500 ppm, particularly preferably from 200 to 500 ppm, of praseodymium (Pr);
from 1 to 1000 ppm, preferably from 1 to 100 ppm, particularly preferably from 5 to 50 ppm, of neodymium (Nd);
from 0 to 20 ppm, preferably from 1 to 10 ppm, of titanium (Ti);
from 0 to 1000 ppm, preferably from 1 to 100 ppm, particularly preferably from 1 to 50 ppm, of calcium (Ca);
from 0 to 100 ppm, preferably from 1 to 10 ppm, of chlorine;
from 0 to 10 000 ppm, preferably from 1 to 8000 ppm, of nitrate.

In particular, the invention provides a process for producing a dehydrogenation catalyst as described, wherein manganese(IV) oxide (MnO₂) is used as at least one manganese compound. In particular, it is possible to use a manganese dioxide having the following composition, where the figures indicate the amount of element or compound based on the total amount of manganese dioxide:
from 95 to 99.99% by weight, preferably from 98 to 99.99% by weight, of MnO₂;
from 0 to 0.5% by weight, preferably from 0.01 to 0.2% by weight, of iron (Fe).

In particular, the invention provides a process for producing a dehydrogenation catalyst as described, wherein titanium dioxide (TiO₂) is used as at least one titanium compound.

In particular, the invention provides a process for producing a dehydrogenation catalyst as described, wherein an ammonium heptamolybdate is used as at least one molybdate compound. In particular, it is possible to use an ammonium heptamolybdate having the following composition, where the figures indicate the amount of element or compound based on the total amount of ammonium heptamolybdate:
from 80 to 85% by weight of MoO₃;
from 0 to 1000 ppm, preferably from 1 to 500 ppm, particularly preferably from 1 to 200 ppm, of potassium (K);
from 0 to 1000 ppm, preferably from 1 to 200 ppm, particularly preferably from 1 to 100 DOM, of sodium (Na).

In particular, the invention provides a process for producing a dehydrogenation catalyst as described, wherein magnesium oxide (MgO) or calcium hydroxide (Ca(OH)₂) or both is/are used as at least one alkaline earth metal compound. In particular, it is possible to use a magnesium oxide having the following composition:
from 92 to 95% by weight of MgO;
from 0 to 3% by weight, preferably from 0.1 to 2% by weight, of calcium (Ca);
from 0 to 2% by weight, preferably from 0.01 to 0.5% by weight, of silicon (Si);
from 0 to 2% by weight, preferably from 0.1 to 1% by weight, of iron (Fe).

In particular, it is possible to use a calcium hydroxide having the following composition:
from 70 to 90% by weight, preferably from 70 to 75% by weight, of CaO;
from 0 to 2% by weight, preferably from 0.1 to 1% by weight, of magnesium (Mg).

The figures relate to the amount of element or compound based on the total amount of the raw material.

To produce the catalyst premix, the components (typically in the form of solid powders) are generally mixed and then mixed with a solvent, in particular water, optionally with addition of a binder. Mixing is preferably carried out by intimate mixing, e.g. by kneading, in a stirred vessel, Mix-Muller, mixer, kneader or extruder, preferably in a Mix-Muller, kneader or mixer.

Shaped catalyst bodies are then typically produced (e.g. by extrusion or pressing) from the catalyst premix obtained in this way and these are subsequently dried and optionally calcined.

The at least one iron compound, the at least one potassium compound, the at least one cerium compound, the at least one manganese compound and the at least one titanium compound (typically in the form of solid powders) are optionally firstly mixed with further metal compounds (in particular at least one alkaline earth metal compound, at least one molybdenum compound) and then mixed with the solvent and optionally at least one binder.

A solvent use is made of, in particular, water or a mixture of polar solvents (e.g. alcohols, esters) and water. As binder (also known as plasticizer), it is possible to use, for example, alginate, starch, carboxymethylcellulose, hydroxyethylcellulose and polyvinyl alcohol. The binders are typically used in the form of a solution in water.

The production of shaped catalyst bodies from the catalyst premix is typically carried out by extrusion or pressing (tablet pressing). Examples of shaped catalyst bodies are cylinders (pellets), rings, star bodies and honeycomb bodies. The production of shaped catalyst bodies from the catalyst premix obtained in step i) is preferably carried out by means of extrusion.

After shaping, the moist shaped bodies are typically dried at temperatures of from 50° C. to 500° C., preferably from 80 to 350° C. Drying can take place for example in a drying oven (e.g. on metal sheets), in a drying drum or on belt dryers.

The shaped catalyst bodies are preferably calcined at temperatures in the range from 500° C. to 1200° C., preferably from 750 to 1000° C., preferably from 800 to 900° C., in step iii). The calcinations is preferably carried out in a rotary furnace.

In a preferred embodiment, the above-described process of the invention for producing a dehydrogenation catalyst comprises the following steps:
i) production of a catalyst premix by mixing at least one iron compound, at least one potassium compound, at least one cerium compound, from 0.7 to 10% by weight, based on the finished catalyst, of at least one manganese compound, calculated as MnO₂, from 10 to 200 ppm, based on the finished catalyst, of at least one titanium compound, calculated as TiO₂, optionally further metal compounds, optionally further components and optionally at least one binder with water;
ii) production of shaped catalyst bodies by extrusion from the catalyst premix obtained in step i);
iii) drying of the shaped catalyst bodies at temperatures in the range from 50° C. to 500° C. and calcination of the shaped catalyst bodies in the range from 500 to 1200° C., preferably from 750 to 1000° C., preferably from 800 to 900° C.

The present invention preferably provides a process for producing a dehydrogenation catalyst as described above, wherein from 0.7 to 10% by weight of manganese dioxide (MnO₂), preferably from 0.7 to 5% by weight, particularly preferably from 0.7 to 3% by weight, in particular from 0.7 to 2% by weight, in particular from 1 to 2% by weight, (based on the finished catalyst) of manganese dioxide (MnO₂) and from 10 to 200 ppm, preferably from 30 to 150 ppm, particularly preferably from 50 to 120 ppm, in particular from 60 to 100 ppm, very particularly preferably from 60 to 80 ppm, (based on the finished catalyst) of titanium dioxide (TiO₂), calculated as TiO₂, are added in step i).

In a further aspect, the present invention provides a process for the catalytic dehydrogenation of a hydrocarbon, wherein a mixture of steam and at least one hydrocarbon is brought into contact with a dehydrogenation catalyst as described above. The hydrocarbon is preferably ethylbenzene.

In particular, the invention provides a process for the catalytic dehydrogenation of a hydrocarbon, wherein low steam/hydrocarbon ratios are used. The present invention preferably provides a process for the catalytic dehydrogenation of a hydrocarbon, wherein a mixture of steam and at least one hydrocarbon having a molar steam/hydrocarbon ratio in the range from 3 to 7.35; preferably in the range from 4 to 7; in particular in the range from 5 to 6, is used. In particular, the invention provides a process for the catalytic dehydrogenation of ethylbenzene to styrene, wherein a mixture of steam and ethylbenzene having a steam/hydrocarbon weight ratio in the range from 0.5 to 1.25; preferably from 0.7 to 1.2; in particular from 0.85 to 1.1; particularly preferably from 0.9 to 1.0, is used.

In a further aspect, the present invention provides a process for the catalytic dehydrogenation of a hydrocarbon, wherein a mixture of steam and at least one hydrocarbon having a molar steam/hydrocarbon ratio in the range from 3 to 7.35; preferably in the range from 4 to 7; in particular in the range from 5 to 6, is brought into contact with a dehydrogenation catalyst comprising
at least one iron compound, at least one potassium compound, at least one cerium compound and 0.7 to 10% by weight of at least one manganese compound, calculated as MnO₂.

As regards the iron compound, the potassium compound, the cerium compound and the manganese compound, the above-described embodiments, in particular, apply.

The process of the invention using a dehydrogenation catalyst comprising at least one iron compound, at least one potassium compound, at least one cerium compound and from 0.7 to 10% by weight of at least one manganese compound gives an improved styrene yield at low S/HC ratios compared to known processes and dehydrogenation catalysts.

The process described can be the dehydrogenation of alkylaromatic or aliphatic hydrocarbons, preferably the dehydrogenation of alkylaromatic hydrocarbons. The process of the invention for the dehydrogenation of a hydrocarbon can be, for example, the dehydrogenation of ethylbenzene to styrene, of isopropylbenzene: to alpha-methylstyrene, of butene to butadiene or of isoamylene to isoprene. The hydrocarbon is preferably ethylbenzene.

Yields of from 40 to 80%, preferably from 50 to 75%, particularly preferably from 60 to 70%, based on the hydrocarbon used, are typically achieved per pass through the reactor in the catalytic dehydrogenation process of the invention. In particular, in the catalytic dehydrogenation of ethylbenzene, styrene yields of from 40 to 80%, preferably from 50 to 75%, particularly preferably from 60 to 70%, based on the ethylbenzene used, are achieved per pass through the reactor. The yields indicated are in mol %.

The process for the catalytic dehydrogenation of a hydrocarbon is typically carried out at temperatures of from 500 to 650° C. and pressures of from 0.2 to 2 bar absolute.

Furthermore, the present invention provides for the use of a dehydrogenation catalyst as described above for the catalytic dehydrogenation of a hydrocarbon, in particular an alkylaromatic or aliphatic hydrocarbon, preferably an alkylaromatic hydrocarbon. The invention preferably provides for the use of a dehydrogenation catalyst as described above for the catalytic dehydrogenation of a hydrocarbon at a molar steam/hydrocarbon ratio in the range from 3 to 7.35; preferably in the range from 4 to 7; in particular in the range from 5 to 6.

Furthermore, the present invention provides for the use of a dehydrogenation catalyst comprising
at least one iron compound, at least one potassium compound, at least one cerium compound and from 0.7 to 10% by weight of at least one manganese compound, calculated as MnO₂,

for the catalytic dehydrogenation of a hydrocarbon, in particular an alkylaromatic or aliphatic hydrocarbon, preferably an alkylaromatic hydrocarbon, particularly preferably ethylbenzene, at a molar steam/hydrocarbon ratio in the range from 3 to 7.35; preferably in the range from 4 to 7; in particular in the range from 5 to 6.

The figures are explained below:

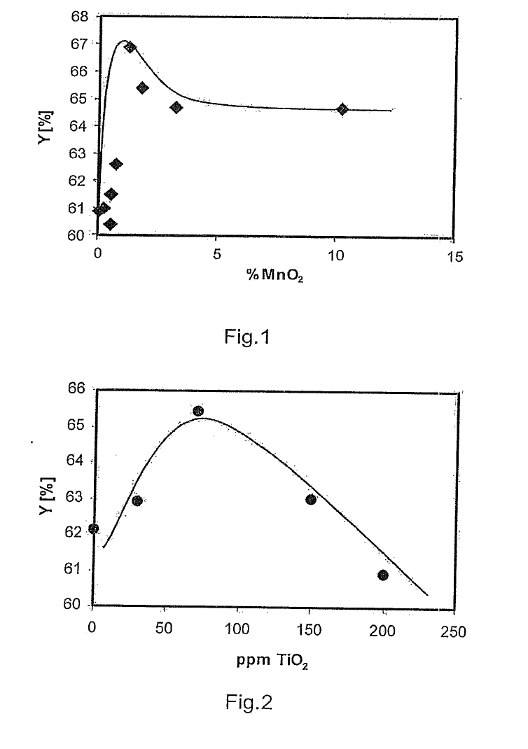

FIG. 1 shows the styrene yield Y in mol % in the catalytic dehydrogenation of ethylbenzene (as per example 7) at a steam/ethylbenzene weight ratio of 1, a temperature of 620° C. and a space velocity of 1.26 ml of ethylbenzene/[(ml of catalyst)·(h)] as a function of the manganese content in % by weight as MnO₂ in the catalyst used (based on the total catalyst), at a titanium content in the catalyst of 70 ppm.

FIG. 2 shows the styrene yield Y in mol % in the catalytic dehydrogenation of ethylbenzene (as per example 7) at a steam/ethylbenzene weight ratio of 1, a temperature of 620° C. and a space velocity of 1.26 ml of ethylbenzene/[(ml of catalyst)·(h)] as a function of the titanium content in ppm in the catalyst used (based on the total catalyst), at a manganese content in the catalyst of 1.8% by weight.

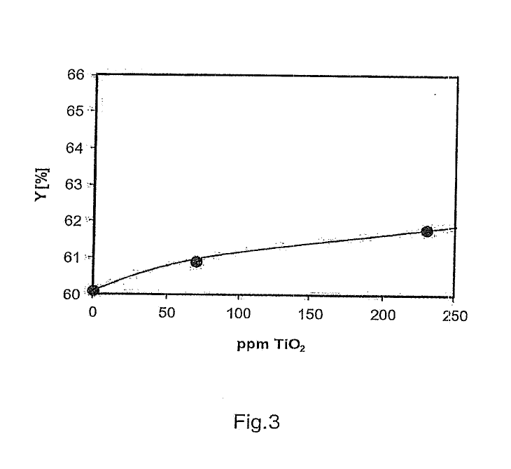

FIG. 3 shows the styrene yield Y in mol % in the catalytic dehydrogenation of ethylbenzene (as per example 7) at a steam/ethylbenzene weight ratio of 1, a temperature of 620° C. and a space velocity of 1.26 ml of ethylbenzene/[(ml of catalyst)(h)] as a function of the titanium content in ppm in the catalyst used (based on the total catalyst), at a manganese content in the catalyst of 0.02% by weight.

The styrene yield in mol % is in each case reported as the molar amount of styrene produced based on the molar amount of ethylbenzene used.

The present invention is illustrated by the following examples.

### EXAMPLES

### Example 1 (Comparative Example) Catalyst A (without Addition of MnO₂ and TiO₂)

An iron oxide Fl (alpha-Fe₂O₃, hematite) comprising 0.027% by weight of manganese (Mn), calculated as MnO₂, and 28 ppm of titanium (Ti), calculated as TiO₂, was used. The BET surface area of the iron oxide Fl was 11 m²/g.

Further components used were potassium carbonate, cerium carbonate, magnesium oxide, calcium hydroxide and ammonium heptamolybdate. The compositions of the raw materials used determined by means of elemental analysis are shown below, where the figures relate to the respective element or the respective compound based on the respective total raw material.
Iron oxide F1: 98.9% by weight of Fe₂O₃; <10 ppm of chlorine (Cl); 0.40% by weight of sulfur (S); 170 ppm of manganese (Mn); 17 ppm of titanium (Ti); 0.07% by weight of chromium (Cr); 0.02% by weight of calcium (Ca).
Cerium carbonate: 52.80% by weight of CeO₂; 195 ppm of lanthanum (La); 370 ppm of praseodymium (Pr); 17 ppm of neodymium (Nd); <10 ppm of titanium (Ti); 3 ppm of chlorine (Cl); 10 ppm of calcium (Ca); 0.52% by weight of nitrate.
Magnesium oxide: 93.87% by weight of MgO; 1.40% by weight of calcium (Ca); 0.50% by weight of silicon (Si); 0.35% by weight of iron (Fe).
Calcium hydroxide: 72.65% by weight of CaO; 0.35% by weight of magnesium (Mg).
Ammonium heptamolybdate: 82% by weight of MoO₃; 90 ppm of potassium (K); 50 ppm of sodium (Na).

A catalyst A having the nominal oxide composition 72.7% by weight of Fe₂O₃, 13.6% by weight of K₂O, 7.4% by weight of CeO₂, 2.2% by weight of MgO, 2% by weight of CaO and 2.1% by weight of MoO₃, 0.02% by weight of Mn0₂ and 20 ppm of TiO₂ was produced.

For this purpose, the abovementioned pulverulent components were firstly mixed dry and then kneaded with addition of water and starch solution. The catalyst composition was extruded to give pellets having a diameter of 3 mm and dried at 120° C. for 1 hour. The shaped catalyst bodies (pellets) were subsequently calcined in air at 350° C. for 1 hour and 825° C. for 1 hour.

### Example 2 (Comparative Example) Catalyst B (without Addition of MnO₂, with Addition of TiO₂)

A catalyst was produced as described in example 1, but, in contrast to example 1, titanium dioxide (TiO₂) was additionally added. The iron oxide F1 was used.

A catalyst B having the nominal oxide composition of 72.7% by weight of Fe₂O₃, 13.6% by weight of K₂O, 7.4% by weight of CeO₂, 2.2% by weight of MgO, 2% by weight of CaO, 2.1% by weight of MoO₃, 0.02% by weight of MnO₂ and 70 ppm (mg/kg) of TiO₂ was obtained.

### Example 3 (Comparative Example) Catalyst C (Iron Oxide with a Proportion of Ti)

A catalyst C was produced without addition of MnO₂ and TiO₂ and using an iron oxide F₂ (Fe₂O₃, hematite) comprising 0.025% by weight of manganese (Mn), calculated as MnO₂, and a proportion of titanium of 195 ppm (mg/kg) (corresponds to 325 ppm of Ti, calculated as TiO₂). The BET surface area of the iron oxide F2 was 2.3 m²/g.

The composition of the iron oxide F2 was determined by means of elemental analysis and is shown below, where the figures relate to the respective element or compound based on the total raw material.
Iron oxide F2: 99.4% by weight of Fe₂O₃; <10 ppm of chlorine (Cl); 0.16% by weight of sulfur (S); 160 ppm of manganese (Mn); 195 ppm of titanium (Ti); 0.0:2% by weight of chromium (Cr).

The production of the catalyst was carried out as described in example 1 using further raw materials described in example 1.

A catalyst C having the nominal oxide composition 72.7% by weight of Fe₂O₃, 13.6% by weight of K₂O, 7.4% by weight of CeO₂, 2.2% by weight of MgO, 2% by weight of CaO, 2.1% by weight of MoO₃, 0.02% by weight of MnO₂ and 240 ppm of TiO₂ was obtained.

Example 4 Catalysts D-J (different additions of manganese) A series of catalysts D, E, F, G, H, I, J were produced with addition of various amounts of MnO₂. The titanium content was set to a constant 70 ppm in these catalysts. An iron oxide F3 (Fe₂O₃, hematite), comprising 0.27% by weight of Mn (calculated as MnO₂) and traces of Ti (<17 ppm as TiO₂), was used here. The BET surface area of the iron oxide F3 was 1.2 m²/g.

Furthermore, potassium carbonate, cerium carbonate, magnesium oxide, calcium hydroxide, ammonium heptamolybdate, manganese dioxide and titanium dioxide were used in such amounts that catalysts having the actual oxide compositions as shown in table 1 were obtained. Unless indicated otherwise, the raw materials described in example 1 were used.

The compositions of iron oxide F3 and the manganese dioxide used were determined by means of elemental analysis and are shown below, where the figures relate to the respective element or the respective compound based on the respective total raw material.
Iron oxide F3: 99.6% by weight of Fe₂O₃; 280 ppm of chlorine (Cl); <0.01% by weight of sulfur (S); 0.17% by weight of manganese (Mn); <10 ppm of titanium (Ti); <10 ppm of chromium (Cr); <10 ppm of calcium (Ca); 24 ppm of copper (Cu); 50 ppm of sodium (Na); 55 ppm of nickel (Ni); 43 ppm of silicon (Si); 16 ppm of zinc (Zn).
Manganese dioxid: 99.10% by weight of MnO₂; 0.14% by weight of iron (Fe).

The shaped catalyst bodies were produced as described in example 1.

### Example 5: Catalysts K-N (Different Additions of Titanium)

A series of catalysts K, L, M, N were produced with addition of various amounts of TiO₂. The manganese content was set to a constant 1.8% by weight (calculated as MnO₂) in these catalysts. The iron oxide F3 (Fe₂O₃, hematite), comprising 0.27% by weight of Mn (calculated as MnO₂) and no Ti (<17 ppm calculated as TiO₂), described in example 4 was used here.

Furthermore, potassium carbonate, cerium carbonate, manganese oxide, calcium hydroxide, ammonium heptamolybdate, manganese dioxide and titanium dioxide were used in such amounts that catalysts having the actual oxide compositions as shown in table 1 were obtained.

The shaped catalyst bodies were produced as described in example 1. Unless indicated otherwise, the raw materials described in example 1 were used.

### Example 6: Catalyst O

A catalyst 0 was produced with addition of 70 ppm of TiO₂ and using an iron oxide F4 comprising no titanium (<17 ppm as TiO₂) and 0.6% by weight of Mn (calculated as MnO₂). The BET surface area of the iron oxide F4 was 1.1 m²/g. The production of the catalyst was carried out as described in example 2 with addition of TiO₂ as titanium source. The further raw materials described in example 1 were used.

The composition of the iron oxide F4 was determined by means of elemental analysis and is shown below, where the figures relate to the respective element or the respective compound based on the total raw material.
Iron oxide F4: 99.4% by weight of Fe₂O₃; 63 ppm of chlorine (Cl); <0.01% by weight of sulfur (S); 0.39% by weight of manganese (Mn); <10 ppm of titanium (Ti); <10 ppm of chromium (Cr); <10 ppm of calcium (Ca); 30 ppm of copper (Cu); 40 ppm of sodium (Na); 100 ppm of nickel (Ni); 36 ppm of silicon (Si); 40 ppm of zinc (Zn).

Furthermore, potassium carbonate, cerium carbonate, manganese oxide, calcium hydroxide, ammonium heptamolybdate and titanium dioxide were used in such amounts that a catalyst having the nominal oxide composition 72.7% of Fe₂O₃, 13.6% of K₂O, 7.4% of CeO₂, 2.2% of MgO, 2% of CaO, 2.1% of MoO₃, 0.5% by weight of MnO₂ and 70 ppm of TiO₂ was obtained. Unless indicated otherwise, the raw materials from example 1 were used.

The compositions of the catalysts were checked by means of elemental analysis and are shown in table 1.

TABLE 1
Compositions of all catalysts (% by weight as oxide).

| | | K₂O | CeO₂ | MgO | CaO | MoO₃ | MnO₂ | |
|---|---|---|---|---|---|---|---|---|
| | Fe₂O₃ % by | % by | % by | % by | % by | % by | % by | TiO₂ |
| Catalyst | weight | weight | weight | weight | weight | weight | weight | ppm |
| A | 72.7 | 13.6 | 7.4 | 2.2 | 2 | 2.1 | 0.02 | 20 |
| B | 72.7 | 13.6 | 7.4 | 2.2 | 2 | 2.1 | 0.02 | 70 |
| C | 72.7 | 13.6 | 7.4 | 2.2 | 2 | 2.1 | 0.02 | 240 |
| D | 72.5 | 13.6 | 7.4 | 2.2 | 2 | 2.1 | 0.2 | 70 |
| E | 72.2 | 13.6 | 7.4 | 2.2 | 2 | 2.1 | 0.5 | 70 |
| F | 72.0 | 13.6 | 7.4 | 2.2 | 2 | 2.1 | 0.7 | 70 |
| G | 71.5 | 13.6 | 7.4 | 2.2 | 2 | 2.1 | 1.2 | 70 |
| H | 71.2 | 13.6 | 7.4 | 2.2 | 2 | 2.1 | 1.8 | 70 |
| I | 69.5 | 13.6 | 7.4 | 2.2 | 2 | 2.1 | 3.2 | 70 |
| J | 62.5 | 13.6 | 7.4 | 2.2 | 2 | 2.1 | 10.2 | 70 |
| K | 71.2 | 13.6 | 7.4 | 2.2 | 2 | 2.1 | 1.8 | 0 |
| L | 71.2 | 13.6 | 7.4 | 2.2 | 2 | 2.1 | 1.8 | 30 |
| M | 71.2 | 13.6 | 7.4 | 2.2 | 2 | 2.1 | 1.8 | 150 |
| N | 71.2 | 13.6 | 7.4 | 2.2 | 2 | 2.1 | 1.8 | 200 |
| O | 72.2 | 13.6 | 7.4 | 2.2 | 2 | 2.1 | 0.5 | 70 |

### Example 7: Dehydrogenation of Ethylbenzene to Styrene at an S/HC of 1.0

The catalysts A to O from examples 1 to 6 were used in the dehydrogenation of ethylbenzene to styrene in the presence of steam. 13.3 ml of catalyst were installed in an isothermal tube reactor. At 620° C. and 1 atm outlet pressure, the catalyst was supplied continuously with 14.6 g/h of ethylbenzene and 14.6 g/h of deionized (DI) water, corresponding to an S/HC weight ratio of 1.0. After stabilization (after about 40 hours), the yield of styrene was determined by gas chromatography. The results in respect of ethylbenzene conversion, styrene selectivity and styrene yield for the various catalysts are shown in table 2 and FIGS. 1 and 2.

Ethylbenzene conversion, styrene selectivity and styrene yield were determined by means of the following formulae:

Conversion (mol %)=[(A*M_{f}−B*Mₚ)/(A*M_{f})]×100

Selectivity (mol %)=[(D*Mₚ−C*M_{f})/(A*M_{f}−B*Mₚ)]×(M_{EB}/M_{ST})×100

Yield (mol %)=conversion×selectivity/100

where:
A: ethylbenzene concentration at the reactor inlet (% by weight)
B: ethylbenzene concentration at the reactor outlet (% by weight)
C: styrene concentration at the reactor inlet (% by weight)
D: styrene concentration at the reactor outlet (% by weight)
M_{f}: average molar mass of the organic starting materials
Mₚ: average molar mass of the organic products
M_{EB}: molar mass of ethylbenzene
M_{ST}: molar mass of styrene

The above figures in respect of concentration and molar masses are in each case based on the organic phase (without water).

FIG. 1 shows the dependence of the styrene yield on the proportion of manganese (Mn) in the dehydrogenation catalyst used. The values relate to catalysts which each have a constant proportion of titanium of 70 ppm (calculated as TiO₂) and to an S/HC weight ratio in the dehydrogenation of 1. It can clearly be seen that an improved yield is obtained at and above a proportion of manganese in the catalyst of at least 0.7% by weight. A particularly high yield of styrene can be achieved using catalysts having a manganese content in the range from 0.7 to 10% by weight, in particular from 0.7 to 5% by weight.

FIG. 2 shows the dependence of the styrene yield on the proportion of titanium (Ti) in the dehydrogenation catalyst used. The values relate to catalysts which each have a constant proportion of manganese of 1.8% by weight in total (calculated as MnO₂) and to an S/HC weight ratio in the dehydrogenation of 1. It can clearly be seen that an optimized styrene yield is obtained at a titanium content in the range from 10 to 200 ppm, in particular from 30 to 150 ppm, in particular from 50 to 100 ppm.

FIG. 3 shows the dependence of the styrene yield on the proportion of titanium (Ti) in the dehydrogenation catalyst used. The values relate to catalysts which each have a constant proportion of manganese of 0.02% by weight in total (calculated as MnO₂) and to an S/HC weight ratio in the dehydrogenation of 1. It was able to be shown that only a significantly lower increase in the yield can be achieved by addition of titanium at an S/HC ratio of 1 and in the case of catalysts having a low manganese content. In contrast, the positive influence of the manganese content of at least 0.7% by weight can be improved further by appropriate selection of the titanium content.

TABLE 2
Results on the dehydrogenation of ethylbenzene at an S/HC weight ratio
of 1 and 620° C.

| | | | | Conversion | | |
|---|---|---|---|---|---|---|
| | Mn | Mn | | of | | Yield of |
| | addition | actual | | ethylbenzene | Selectivity to | styrene |
| | % | % | TiO₂ | (EB) | styrene | Mol |
| Catalyst | oxide | oxide | ppm | mol % of EB | mol % of ST | % of ST |
| A | 0 | 0.02 | 0 | 62.7 | 95.9 | 60.1 |
| B | 0 | 0.02 | 70 | 63.8 | 95.5 | 60.9 |
| C | 0 | 0.02 | 240 | 64.0 | 96.5 | 61.8 |
| D | 0 | 0.2 | 70 | 63.6 | 95.9 | 61.0 |
| E | 0.3 | 0.5 | 70 | 64.1 | 95.9 | 61.5 |
| F | 0.5 | 0.7 | 70 | 65.3 | 95.9 | 62.6 |
| G | 1.0 | 1.2 | 70 | 70.2 | 95.2 | 66.9 |
| H | 1.6 | 1.8 | 70 | 68.7 | 95.2 | 65.4 |
| I | 3 | 3.2 | 70 | 67.8 | 95.4 | 64.7 |
| J | 10 | 10.2 | 70 | 67.7 | 95.6 | 64.7 |
| K | 1.6 | 1.8 | 0 | 64.9 | 95.6 | 62.1 |
| L | 1.6 | 1.8 | 30 | 65.8 | 95.6 | 62.9 |
| M | 1.6 | 1.8 | 150 | 65.7 | 95.9 | 63.0 |
| N | 1.6 | 1.8 | 200 | 63.3 | 96.1 | 60.9 |
| O | 0 | 0.50 | 70 | 62.8 | 96.2 | 60.4 |

### Example 8: Dehydrogenation of Ethylbenzene to Styrene at an S/HC of 1.25

The catalysts A to O from examples 1 to 6 were used in the dehydrogenation of ethylbenzene to styrene in the presence of steam, as described in example 7, with an S/HC weight ratio of 1.25 being set. 13.3 ml of catalyst were installed in an isothermal tube reactor. At 620° C. and 1 atm outlet pressure, the catalyst was supplied continuously with 14.6 g/h of ethylbenzene and 18.25 g/h of deionized (DI) water, corresponding to an S/HC weight ratio of 1.25.

The results in respect of ethylbenzene conversion, styrene selectivity and styrene yield for the various catalysts are shown in table 3. In addition, table 3 shows the decreases in the styrene yields when changing from an S/HC weight ratio of 1.25 to an S/HC weight ratio of 1.0 (delta Y 1.25→1.0).

TABLE 3
Results on the dehydrogenation of ethylbenzene at an S/HC weight
ratio of 1.25 and 620° C.

| | | | | Conversion | | | |
|---|---|---|---|---|---|---|---|
| | Mn | Mn | | of | | Yield of | |
| | addition | actual | | ethylbenzene | Selectivity | styrene | Delta Y |
| | % | % | TiO₂ | (EB) | to styrene | mol % of | 1.25→1.0 |
| Catalyst | oxide | oxide | ppm | mol % of EB | mol % of ST | ST | % |
| A | 0 | 0.02 | 0 | 71.4 | 95.4 | 68.1 | 8.0 |
| B | 0 | 0.02 | 70 | 70.4 | 96.1 | 67.6 | 6.7 |
| C | 0 | 0.02 | 240 | 73.5 | 95.7 | 70.3 | 8.5 |
| D | 0 | 0.2 | 70 | 68.5 | 95.7 | 65.6 | 4.6 |
| E | 0.3 | 0.5 | 70 | 72.9 | 95.5 | 69.6 | 8.1 |
| F | 0.5 | 0.7 | 70 | 71.6 | 95.6 | 68.5 | 5.9 |
| G | 1.0 | 1.2 | 70 | 73.1 | 95.3 | 69.7 | 2.8 |
| H | 1.6 | 1.8 | 70 | 73.2 | 95.0 | 69.5 | 4.1 |
| I | 3 | 3.2 | 70 | 72.7 | 95.2 | 69.2 | 4.5 |
| J | 10 | 10.2 | 70 | 72.1 | 95.6 | 68.9 | 4.2 |
| K | 1.6 | 1.8 | 0 | 70.0 | 95.4 | 66.8 | 4.7 |
| L | 1.6 | 1.8 | 30 | 70.8 | 95.5 | 67.6 | 4.7 |
| M | 1.6 | 1.8 | 150 | 72.3 | 95.6 | 69.1 | 6.1 |
| N | 1.6 | 1.8 | 200 | 71.7 | 97.2 | 69.7 | 8.8 |
| O | 0 | 0.50 | 70 | 71.0 | 95.9 | 68.1 | 7.7 |

The improved stability of the catalyst is additionally shown by a smaller loss of catalyst activity when changing from a medium to a low S/HC ratio (comparison of the results of examples 7 and 8).

### Example 9: Dehydrogenation of Butene to Butadiene

The catalyst H was used in the dehydrogenation of 1-butene to butadiene. A volume of 38 ml of catalyst was used in an isothermally heated tube reactor. At 620° C. and 1 atm, the reactor was continuously supplied with 60 g/h of deionized water and 22.5 g/h of 1-butene (corresponding to a molar ratio of steam/butene of 8.6). After stabilization (about 16 hours), the product mixture obtained was analyzed by gas chromatography. The conversion of 1-butene and the butadiene selectivity were calculated using the formulae as in example 7, replacing ethylbenzene by 1-butene and styrene by butadiene. The butene conversion was 23.3 mol % and the butadiene selectivity was 91.2 mol %.

1-16. (canceled)

17. A process for catalytic dehydrogenation of a hydrocarbon, wherein a mixture of steam and at least one hydrocarbon having a molar steam/hydrocarbon ratio in the range from 3 to 7.35 is brought into contact with a dehydrogenation catalyst comprising
at least one iron compound, at least one potassium compound, at least one cerium compound and from 0.7 to 10% by weight of at least one manganese compound, calculated as MnO₂.

18. The catalytic dehydrogenation process according to claim 1, wherein a mixture of steam and at least one hydrocarbon having a molar steam/hydrocarbon ratio in the range from 4 to 7 is used.

19. The catalytic dehydrogenation process according to claim 1, wherein the hydrocarbon is ethylbenzene.

20. The catalytic dehydrogenation process according to claim 1, wherein the dehydrogenation catalyst comprises at least one iron compound, at least one potassium compound, at least one cerium compound, from 0.7 to 10% by weight of at least one manganese compound, calculated as MnO₂, and from 10 to 200 ppm of at least one titanium compound, calculated as TiO₂.

21. The catalytic dehydrogenation process according to claim 1, wherein the dehydrogenation catalyst comprises from 0.7 to 3% by weight of at least one manganese compound, calculated as MnO₂.

22. The catalytic dehydrogenation process according to claim 1, wherein the dehydrogenation catalyst comprises from 30 to 150 ppm of at least one titanium compound, calculated as TiO₂.

23. The catalytic dehydrogenation process according to claim 1, wherein the dehydrogenation catalyst comprises
from 50 to 90% by weight of at least one iron compound, calculated as Fe₂O₃;
from 1 to 30% by weight of at least one potassium compound, calculated as K₂O;
from 0.7 to 10% by weight of at least one manganese compound, calculated as MnO₂;
from 10 to 200 ppm of at least one titanium compound, calculated as TiO₂;
from 2 to 20% by weight of at least one cerium compound, calculated as CeO₂; and
optionally from 0 to 30% by weight of at least one further component.

24. The catalytic dehydrogenation process according to claim 1, wherein the dehydrogenation catalyst comprises from 0.1 to 10% by weight of at least one compound selected from the group consisting of molybdenum, tungsten and vanadium, calculated as oxide in the respective highest oxidation state, as further component.

25. The catalytic dehydrogenation process according to claim 1, wherein the dehydrogenation catalyst comprises from 0.1 to 10% by weight of at least one alkaline earth metal compound, calculated as oxide, as further component.

26. The catalytic dehydrogenation process according to claim 1, wherein the dehydrogenation catalyst comprises
from 50 to 90% by weight of at least one iron compound, calculated as Fe₂O₃;
from 1 to 30% by weight of at least one potassium compound, calculated as K₂O;
from 0.7 to 10% by weight of at least one manganese compound, calculated as MnO₂;
from 10 to 200 ppm of at least one titanium compound, calculated as TiO₂;
from 2 to 20% by weight of at least one cerium compound, calculated as CeO₂;
from 0.1 to 10% by weight of at least one magnesium compound, calculated as MgO;
from 0.1 to 10% by weight of at least one calcium compound, calculated as CaO;
from 0.1 to 10% by weight of at least one molybdenum compound, calculated as MoO₃;
from 0 to 10% by weight of at least one vanadium compound, calculated as V₂O₅, and
from 0 to 10% by weight of at least one further component.
